(19) United States
(12) Patent Application Publication
(10) Pub. No.: US 2011/0150808 A1
(43) Pub. Date: Jun. 23, 2011

(54) METHOD FOR MANUFACTURE OF PIGMENT-CONTAINING COSMETIC COMPOSITIONS AND RELATED COMPOSITIONS

(76) Inventor: RICHARD RIGG, Springfield Gardens, NY (US)

(21) Appl. No.: 12/973,355

(22) Filed: Dec. 20, 2010

Related U.S. Application Data
(60) Provisional application No. 61/288,188, filed on Dec. 18, 2009.

(51) Int. Cl. A61K 8/92; C08K 3/22; C08K 5/52; C08K 5/16; C08L 67/00; A61Q 1/10

(52) U.S. Cl. 424/70.7; 524/401; 524/140; 524/251; 524/503; 524/603

(57) ABSTRACT

The present invention relates to pigment colorants and more directly to a pigment composition comprising a pigment treated with a water-dispersible polyester or polyesteramide polymer in the presence of added water. The invention also relates to a method grinding a mixture containing a pigment with a water-dispersible polyester or polyesteramide in the presence of added water. Such pigment compositions are particularly useful as the colorant in cosmetic composition

### CROSS REFERENCE TO RELATED APPLICATIONS

This application claims priority to U.S. Ser. No. 61/288,188 filed Dec. 18, 2009, the entire contents of which are herein incorporated fully by reference.

### BACKGROUND OF THE INVENTION

1. Field of the Invention

The present invention relates to a method for making cosmetic compositions and the cosmetic composition itself. More particularly, the invention relates to a method for making pigment-containing cosmetics by coating pigments with a water dispersible polymer. The present invention further relates to a pigment composition comprising a colorant pigment treated with a water-dispersible polyester or polyesteramide polymer in the presence of added water and a method for making the same. Finally and further, the invention also relates to a method of preparing a mixture containing a pigment with a water-dispersible polyester or polyester amide in the presence of added water.

2. Description of the Related Art

The related art involves the manufacture of color cosmetics, such as foundations, blushes, eyeshadows, lipsticks, mascaras as well as other colored materials, such as inks. This manufacture necessarily involves a step of incorporating pigments into the formulation.

When the final product has a liquid, particularly aqueous, base, the incorporation of the pigment is problematic and difficult because the most commonly used pigments, such as titanium, iron oxides, or carbon black are insoluble particles which do not easily blend into the liquid base. These particles can be physically dispersed in water. Once incorporated, however a further problem arises, namely, particles can tend to re-agglomerate due to the natural incompatibility of the pigments with water in the formulation; particles can also be adversely affected by the liquid environment, resulting in a loss of color stability.

These concerns give rise to a product which is non-uniform, and unacceptable both to the manufacturer and to the average consumer.

In an effort to enhance the dispersibility of pigment particles and to hinder tendencies for re-agglomeration, the industry has turned to coating of pigment particles in a limited way.

Pigment coatings are chosen for their ability to render the coated particles more compatible with the environment in which they are to be used. Such coating materials are well-known, and have been described for example in U.S. Pat. Nos. 4,349,389; 5,013,543; 3,546,008; 3,734,874; 3,779,993; and 4,233,196, the entire contents and teaching of each of which are fully incorporated herein by reference.

The coatings described in the latter four references, which are polyesters and polyesteramides, are useful for preparing water-dispersible pigments. However, the recommended method for preparation and dispersion of the coated pigments is a wet process, using relatively small quantities of coating and at least 5% water. Although to some extent effective, this methodology can result in incomplete coating of the pigment, which can ultimately permit re-aggregation of the pigments.

This wet preparation process also results, even after drying, in a pigment which does not store well for long periods of time and may be subject to bacterial contamination and raise substantial product safety concerns. U.S. Pat. No. 5,993,834, the entire contents of which are fully incorporated herein) also uses coatings which are polyesters and polyesteramides, polyols such as 1,3 butylenes glycol and ethoxylated surfactants and no water for dispersion of iron oxides. The recommended composition does not utilize water or the more effective ionic surfactants. Although somewhat effective, this composition is strongly limiting because water is required to effectively allow ionic surfactants to wet and disperse pigments. Therefore, the '834 contents are greatly limited in that the disclosure focuses on iron oxides and does not cover the more difficult organic pigment such as carbon black which is used in cosmetics and are very difficult to disperse.

Accordingly, there is a need for an improved method and composition that overcomes at least one of these above-noted difficulties.

### ASPECTS AND SUMMARY OF THE INVENTION

An aspect of the present invention is to provide a pigment containing composition and an optional method for making the same.

Another aspect of the present invention is to provide a treated pigment which is more effectively coated especially for carbon black pigments, stable even after prolonged storage time, and which disperses easily, and without re-agglomeration, in an aqueous base.

Another aspect of the present invention is to provide a method of grinding a mixture containing a pigment with a water-dispersible polyester or polyesteramide in the presence of added water.

The present invention also relates to a pigment composition comprising a pigment treated with a water-dispersible polyester or polyesteramide polymer in the presence of added water. The invention also relates to a method of grinding a mixture containing a pigment with a water-dispersible polyester or polyesteramide in the presence of added water. Such pigment compositions are particularly useful as the colorant in cosmetic composition.

The present invention relates also to pigment composition comprising pigment particles treated with a water-dispersible polyester or polyesteramide in the presence of water and ionic surfactants and an alkalizing agent. The invention also comprises a method for manufacturing the pigment composition comprising co-grinding pigment and the water-dispersible polymers in low levels of water, surfactants. Also provided are cosmetic compositions comprising the pigment composition.

According to an alternative and optional embodiment of the present invention there is provided a pigment composition, comprising: a pigment treated with at least one of a water-dispersible polyester, polyesteramide polymer or Polyvinylpyrolidone (PVP) or Cellulosic polymers that are natural or synthetic acrylic polymers, an alkalizing agent and a plasticizer in the presence of added water and a preservative.

According to another alternative and optional embodiment of the present invention, there is provided a pigment composition, comprising: pigment in an amount of about 5-80%, treated with a water-dispersible polyester or polyesteramide having a dicarboxylic acid component, a sulfomonomer component, and polymer or PVP, or cellulosic polymer or acrylic polymers and a pH adjusting agent, in presence of added water.

According to another alternative and optional aspect of the present invention, there is provided a pigment composition which comprises: one of an organic or inorganic pigment in an amount of about 5-80%, treated with a water-dispersible polyester or polyesteramide polymer having an aromatic dicarboxylic acid moiety, an aliphatic or cycloaliphatic glycol residue, an average, per molecule, of between five and eight sodiosulfo(SO₃⁻Na₊) substituents on the aromatic dicarboxylic acid units, in an amount of about 20-95%, a surfactant in an amount of about 0.1-25% and ethoxylated glycol in an amount of from about 0.1-10%, in the presence of 0.2-4.99% of water.

According to another alternative and optional aspect of the present invention, there is provided a method of making a pigment composition comprising the steps of: grinding a mixture containing a pigment with a water-dispersible polyester or polyesteramide, or Polyvinylpyrolidone (PVP) or Cellulosic polymers that are natural or synthetic acrylic polymers, an alkalizing agent and a plasticizer in the presence of added water.

The above, and other aspects, features and advantages of the present invention are to be understood as alternatives to the proposed invention and not limiting among themselves, as will become apparent from the following description read in conjunction with the accompanying tables, in which like identifiers designate the same elements.

### BRIEF DESCRIPTION OF THE DRAWINGS

None

### DETAILED DESCRIPTION OF THE PREFERRED EMBODIMENTS

The preferred water-dispersible coatings are polyesters and polyesteramides, such as are available from Eastman Chemical Corporation, under the trade name Eastman AQ Polymers. These polymers are described, for example, in U.S. Pat. Nos. 3,546,008; 3,734,874; 3,779,993; and 4,233,196, the contents of which are incorporated herein fully by reference for the understanding of those of skill in the art.

Pigment particles pre-coated with these polymers are commercially available from Eastman, and have been described, for example, in U.S. Pat. Nos. 5,260,052; 5,143,671, and 5,055,500, also incorporated herein fully by reference. Their use in cosmetic compositions has also previously been described, for example, in U.S. Pat. No. 5,169,881 and WO 96/03964, which contents are also fully incorporated herein by reference.

Unfortunately, in each of the above general references regarding polymers or coatings, the coated pigment compositions are prepared by a “wet” process: the pigment and coating polymer are combined together in the presence of a significant quantity, i.e., from 5 up to 50%, of water in the total starting mixture. Although the pigment composition so formed can be subsequently dried, this process mandates that some amount of water will remain in the formulation.

U.S. Pat. No. 5,993,834 also uses coatings which are polyesters and polyesteramides, polyols such as 1,3 butylenes glycol and ethoxylated surfactants and no water for dispersion of iron oxides. The recommended composition does not utilize water or the more effective ionic surfactants but has other severe disadvantages. Although somewhat effective, this composition is limiting because water is required to effectively allow ionic surfactants to wet and disperse pigments. The invention is limiting in that it is focused on iron oxides and does not cover the more difficult organic pigment such as carbon black which is widely used in cosmetics and are very difficult to disperse.

In contrast, the pigment composition of the present invention is prepared by a process that utilizes extremely small amounts of water, surfactants, and pH adjusting agents. In a general sense, the process involves mixing the components: water, surfactants, ph adjusting agent, applying heat to a temperature at or above the T_{g} of the polymers and dispersing on a standard roller mill, to the appropriate particle size. The resulting pigment composition needs no further drying, and can be stored for long periods of time with little threat of bacterial contamination promoted by the presence of residual water in the formulation (there is no residual water).

As noted above, the polyesters and polyesteramides, PVP, cellulose polymers and acrylic polymers useful in the invention are known compounds and commercially available. The polymers of the present invention are such linear water-dispersible polyesters or polyesteramides which are reaction products of the following reactants, or the ester- or esteramide-forming derivatives thereof:
(1) at least one difunctional dicarboxylic acid;
(2) from 2-25 mole % of at least one difunctional sulfomonomer containing at least one metallic sulfonate group or nitrogen-containing non-metallic sulfonate group attached to an aromatic or cycloaliphatic nucleus wherein the functional groups are hydroxy, carboxyl or amino, or a mixture thereof;
(3) at least one difunctional reactant selected from a glycol, or a mixture of a glycol having two —NRH groups, the glycol containing two —CH₂—OH groups; and
(4) from 0-40 mole % of a difunctional reactant selected from hydroxycarboxylic acids having one —C(R)₂—OH group, aminocarboxylic acids having one —NRH group, and amino alcohols having one —C(R)₂—OH group and one —NRH group, of mixtures thereof;

wherein each R of (3) and (4) is H or an alkyl group containing 1 to 4 carbon atoms, and all mole percentages are based on the total of all acid, hydroxyl, and amino equivalents being equal to 200 mole %.

The polymers employed in the pigment composition have an inherent viscosity of at least 0.1, and preferably at least 0.3, and a glass transition temperature of from 25 degrees C. to 90 degrees C. when the polymer is in the dry state. Preferred polymers are those having an dicarboxylic acid component, a difunctional sulfomonomer component, and a glycol component, especially those having an aromatic dicarboxylic acid moiety, an aliphatic or cycloaliphatic glycol residue, and on average, per molecule, between five and eight sodiosulfo(SO₃⁻NA⁺) substituents on the aromatic dicarboxylic acid units. Particularly preferred is the polymer known as AQ 55S, available from Eastman Chemical Company.

According to the discussion herein, below therefore are exemplary pigment or ‘chip’ compositions. The use of any of such compositions provide the following benefits:
(A) Water is needed to solubilize many wetting and dispersing chemicals which when added to the chip making process acts as a wetting and dispersing aid, so that when the final chips are used into a finished product, it will:
   a. dissolve/disperse much easier in the presence of a water soluble wetting and dispersing agent;
   b. the pigment will wet much easier (more rapidly and completely) because of the presence of a water soluble dispersing chemicals; and/or
   c. the color intensity will be far greater because of the water soluble dispersing Chemicals which act to completely wet the pigment throughout the composition or cosmetic composition.

TABLE I
Examples of Chip Composition

| | | Chip 1 | Chip 2 | Chip 3 | Chip 4 | Chip 4 | Chip 5 |
|---|---|---|---|---|---|---|---|
| Trade name | Phase | Wt. % | Wt. % | Wt. % | Wt. % | Wt. % | Wt. % |
| Carbon black | A | 40.0 | 40.0 | 40.0 | 20.0 | 20.0 | 40.0 |
| Black Iron Oxide | | — | — | — | 20.0 | 10.0 | — |
| Eastman Aq 55S | | 50.0 | — | — | 50.0 | 60.0 | — |
| Eastman Aq 38S | | — | 50.0 | — | — | — | — |
| Eastman Aq 48S | | — | — | 50.0 | — | — | — |
| Shellac | | — | — | — | — | — | 50.0 |
| Water | B | 5.0 | 5.0 | 4.0 | 4.5 | 4.0 | 4.0 |
| PVP | | 1.0 | — | 0.5 | 0.5 | 1.0 | 1.0 |
| Polyvinyl Alcohol | | — | 1.0 | — | 0.5 | 0.5 | 0.5 |
| PEG 8000 | | — | 1.0 | 2.0 | — | 0.6 | 0.6 |
| PEG 400 | | — | — | 1.0 | 2.0 | 0.5 | 1.1 |
| Dispersun WS-90 | C | 2.5 | 2.5 | — | 1.0 | 2.0 | 2.0 |
| Sodium Laureth | | — | — | 2.0 | 1.0 | 0.8 | — |
| sulphate | | | | | | | |
| TEA | D | — | — | — | — | 0.6 | 0.2 |
| Sodium Hydroxide | | 0.5 | 0.50 | 0.5 | 0.5 | — | 0.6 |
| 50% solution | | | | | | | |

Regarding Phase A: It will be understood that the Eastman Aq elements can be found in full description at www.eastman.com/product as provided by the Eastman Chemical Company in Kingsport, Tenn. 37662, USA. For example, Eastman Aq 55S polymer has an INCI name of Polyester-5. The remaining elements are commonly available on a commercial basis and multiple sources will be known to those of skill in the art. Regarding Phase B: It will be understood that PVP (Polyvinylpyrolidone), PVA (Polyvinyl Alcohol), and PEG 8000 or PEG 400 will be understood to those of skill in the art. Supplies are commonly available on a commercial basis and multiple sources will be known to those of skill in the art. Regarding Phase C: Dispersun WS-90 (also Dispersun DSP-W90) is a PEG-26-PPG-30 Phosphate and a dispersant available by Innospec at http://www.specialchem4cosmetics.com/product-directory and Sodium Laureth Sulphate is commonly available on a commercial basis and multiple sources will be known to those of skill in the art.

The following is a proposed manufacturing process or method as disclosed by the present inventive description:
Step 1: Select a specific batch composition from Table I above:
Step 2: Prepare Phase A (Eastman Aq polymers and carbon black or other elements) are combined in a heated jacketed mixer with agitation blade and heated to 200 Celsius for 20-60 minutes. Such heated mixers are known to those of skill in the art and are provided to support adequate mixing at a stable temperature.
Step 3: Phase B materials are dissolved in water
Step 4: Phase C ingredients are added to phase B and dissolved
Step 5: Phase D is added to phase BC and mixed
Step 6: Phases BCD are added to phase A and heating to 200 C is maintained for 30 minutes
Step 7: Phases ABCD are then pored on a chill roll and chipped.

In the most preferred embodiment for the present invention, the chips of composition 1 in Table I are preferred, but those of skill in the art will recognize that alternative compositions and methods may be used without departing from the scope and spirit of the present invention.

According to the discussion herein, below therefore are exemplary mascara formulations containing pigment or ‘chip’ compositions. The use of any of such cosmetic composition may be modified without departing from the scope and spirit of the present invention. Table II provides mascara formulations:

TABLE II
Examples of mascara formulations containing pigment chips

| | | Formulations | | | |
|---|---|---|---|---|---|
| | | 1 | 2 | 3 | 4 |
| Phase | Ingredient | Wt % | Wt % | Wt % | Wt % |
| A | Water | 65.90 | 43.4 | 43.6 | 40.5 |
| | HEC HHR | 0.40 | 0.40 | 0.2 | 0.3 |
| | PVP | — | 2.0 | 0.5 | 1.0 |
| | Methyl Paraben | 0.20 | 0.20 | 0.2 | 0.2 |
| | TEA 99% | 2.0 | 2.0 | 1.5 | 2.0 |
| B | Pigment Chips | 10.0 | 10.0 | 8.0 | 5.0 |
| | Black Iron Oxide | — | 3.0 | 2.0 | 2.0 |
| C | Propylene glycol monostearate | 2.0 | 3.0 | 2.5 | 2.0 |
| | Stearic Acid | 3.0 | 3.0 | 3.0 | 3.0 |
| | Lecithin | — | 0.50 | 1.0 | 1.50 |
| | Beeswax | 4.0 | 3.0 | 5.0 | 2.0 |
| | Carnauba wax | 2.0 | 2.5 | 3.0 | 4.0 |
| | Candelilla wax | 2.0 | 3.0 | 4.0 | 5.0 |
| | Polyethylene wax | 6.0 | 6.0 | 3.0 | 5.0 |
| | Sylvatac 85 | — | — | 2.0 | 1.0 |
| | Ganex V220 | — | 1.0 | 3.0 | 4.0 |
| | Ganex V216 | — | 2.0 | 2.0 | 1.0 |
| | Propyl Paraben | 0.20 | 0.2 | 0.2 | 0.2 |
| D | Water | 2.0 | | | |
| | Germall 115 | 0.30 | 0.3 | 0.3 | 0.3 |
| E | Interpolymer PC 5775 | — | 15.0 | — | 10.0 |
| | Interpolymer PC 5227 | — | — | 15.0 | 10.0 |

Those of skill in the cosmetic formulation art will recognize that the generic names for ingredients are commonly available on a commercial basis and multiple sources will be known to those of skill in the art. Those of skill in the cosmetic formulation arts will also recognize that the trade names noted above are also commonly available on a commercial basis and refer to specific compositions provided by the manufacturers under those formulations. For example, Germall 115 is a tradename for a free-flowing hygroscopic powder of INCI, imidazolidinyl Urea found at www.ispcorp.com/product details.com. Similarly, Ganex V220 and Ganex V216 are similarly commercially available. TEA is Triethylamine and is available from numerous commercial sources, as is methyl paraben and propyl paraben, water, PVP, Interpolymer PC 575 and 5227 and HEC HHR, etc.

In one aspect of the present invention, the mascara formulation according to formulation 4 is preferred.

The following is a proposed manufacturing process or method for the cosmetic, as disclosed by the present inventive description:
Step 1: Select a specific composition from Table II above:
Step 2: Combine Phase A cold while mixing.
Step 3: Heat phase A to 85 Celsius (C) while mixing.
Step 4: Maintain 85 C temp and mixing for 30 minutes.
Step 5: Add phase B with mixing and mix until phase B is fully dispersed.
Step 6: In a separate container heat phase C to 85 C until it is melted and homogeneous.
Step 7: Add phase C to phase AB at 80-85 C while mixing.
Step 8: Mix for 20 minutes then begin cooling.
Step 9: Cool to 50 C and add phase D.
Step 10: Add phase E slowly while mixing.
Step 11: Add phase E slowly while mixing.
Step 12: Continue cooling while mixing and drop batch at 32-35 C.

The pigment component of the present composition can be any which is insoluble or sparingly soluble in water. The pigment compositions have utility in virtually any industry in which water dispersible pigments are used, for example, in manufacturing inks or other industrial pigmented product, and the pigment utilized will be chosen accordingly. However, a preferred use of the compositions is for cosmetic applications, and in this case, the pigment is chosen from those which are cosmetically acceptable. In general, the pigments employed may be organic or inorganic. However, it will be apparent that the choice of pigment should preferably not include those having large amounts of bivalent or multivalent ionizable cations which can interfere with the water dispersibility of the polyester component. Choice of pigment should also be made keeping in mind the temperature at which the grinding process will take place, so as to avoid pigments which are not stable at the processing temperature.

Non-limited examples of useful inorganic pigments include iron oxides (yellow, red, brown or black), ferric ammonium ferrocyanide(blue), manganese violet, ultramarine blue, chrome oxide(green), talc, lecithin modified talc, zeolite, kaolin, lecithin modified kaolin, titanium dioxide(white) and mixtures thereof. Other useful pigments are pearlants such as mica, bismuth oxychloride and treated micas, such as titanated micas and lecithin modified micas. Other examples are provided in the tables noted below.

The organic pigments include natural colorants and synthetic monomeric and polymeric colorants. Exemplary are Carbon Black, phthalocyanine blue and green pigment, diarylide yellow and orange pigments, and azo-type red and yellow pigments such as toluidine red, litho red, naphthol red and brown pigments. Also useful are lakes, which are pigments formed by the precipitation and absorption of organic dyes on an insoluble base, such as alumina, barium, or calcium hydrates. Particularly preferred lakes are primary FD&C or D&C lakes and blends thereof.

Also included are copolymer pigments that are water insoluble, e.g., nylon powder, polyethylene, and polyesters. The polyesters can include linear, thermoplastic, crystalline or amorphous materials produced using one or more diols and one or more dicarboxylic acids copolymerized with colorants. Other pigments to be used in the invention will be apparent to one of ordinary skill in the art without departing from the scope and spirit of the present invention.

Those of skill in the art having studied the entire disclosure herein shall recognize that the phrase “polyesteramide” is similarly understood as polyester amide (with the space), without departing from the scope and spirit of the present invention. The use of either form shall be considered proper and understood by one of skill in the art.

The novel features that are considered characteristic of the invention are set forth with particularity in the appended claims. The invention itself, however, both as to compositions and operation and methods together with the additional aspects and advantages thereof will best be understood from the following description of the preferred embodiments of the present invention. If any other non-standard meaning is intended, the specification will specifically state that a special meaning is being applied to a word or phrase. Likewise, the use of the words “function” or “means” in the Description of Preferred Embodiments is not intended to indicate a desire to invoke the special provision of 35 U.S.C. 112, paragraph 6 to define the invention. To the contrary, if the provisions of 35 U.S.C. 112, paragraph 6, are sought to be invoked to define the invention(s), the claims will specifically state the phrases “means for” or “step for” and a function, without also reciting in such phrases any structure, material, or act in support of the function.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted by those of skill in the art as having a meaning that is consistent with their meaning required in the context of the relevant art in a way that will bring life and meaning to the disclosure herein, and will not be interpreted in an idealized or overly formal sense unless expressly so defined herein.

Having described at least one of the preferred embodiments of the present invention with reference to the accompanying drawings, it is to be understood that the invention is not limited to those precise embodiments, and that various changes, modifications, and adaptations may be effected therein by one skilled in the art without departing from the scope or spirit of the invention as defined in the appended claims.

What is claimed is:

1. A pigment composition, comprising:
a pigment component from 85.0-95.0% by weigh, further comprising:
cabon black being 35.0-45.0% by weight and polyester being 45.0-55.0% by weight;
a fluid component, including water, from 5.0-8.5% by weight;
a dispersant component from 1.5-3.0% by weight; and
a sodium hydroxide 50% solution from 0.25-0.75% by weight.

2. A pigment composition, according to claim 1, wherein:
said fluid component is about 5.0% by weight water and about 1.0% by weight Polyvinylpyrolidone (PVP).

3. A pigment composition, according to claim 2, wherein:
said dispersant component is PEG-26-PEG-30 Phosphate of about 2.5% by weight.

4. A pigment composition, according to claim 3, wherein:
said carbon black is about 40.0% by weight and said polyester is about 50.0% by weight.

5. A pigment composition, according to claim 2, wherein:
said dispersant component includes PEG-26-PEG-30 Phosphate and sodium laureth sulphate.

6. A pigment composition, according to claim 1, wherein:
said fluid component includes 4.0-5.0 by water and 0.5-1.0% by weight polyvinyl alcohol.

7. A pigment composition, according to claim 1, further comprising:
Triethylamine (TEA) about 0.10-0.70% by weight.

8. A method of producing a pigment composition, comprising the steps of:
combining, in a first heated and agitated mixer to 200 Celsius for 20-60 minutes, as a phase A:
a pigment component from 85.0-95.0% by weigh, further comprising:
cabon black being 35.0-45.0% by weight and polyester being 45.0-55.0% by weight;
dissolving separately, as a phase B, a fluid component of about 5.0% by weight water and about 1.0% by weight Polyvinylpyrolidone (PVP);
mixing, into phase B, a dispersant component from 1.5-3.0% by weight as a phase C;
mixing, into phase B and C, a sodium hydroxide 50% solution from 0.25-0.75% by weight as a phase D;
combining phase A with said mixed phased B, C, and D and heating to 200 Celsius for 20-40 minutes to form a pigment solution; and
performing a chilling and chipping process on said pigment solution, thereby producing said pigment composition for later use.

9. A pigment composition, comprising:
a pigment treated with at least one of a water-dispersible polyester, polyesteramide polymer or Polyvinylpyrolidone (PVP) or Cellulosic polymers that are natural or synthetic acrylic polymers, an alkalizing agent and a plasticizer in the presence of added water and a preservative.

10. The composition of claim 9, wherein:
the polymer has a dicarboxylic acid component, a sulfomonomer component, and a surfactant component

11. A pigment composition, comprising:
pigment in an amount of about 5-80%, treated with a water-dispersible polyester or polyesteramide having a dicarboxylic acid component, a sulfomonomer component, and polymer or PVP, or cellulosic polymer or acrylic polymers and a pH adjusting agent, in presence of added water; and
the polymer comprises a dicarboxylic acid component, a difunctional sulfomonomer component, and a glycol component.

12. The composition of claim 11, wherein:
the polymer comprises an aromatic dicarboxylic acid moiety, an aliphatic or cycloaliphatic glycol residue, and an average, per molecule, of between five and eight sodiosulfo(SO₃⁻Na⁺) substituents on the aromatic dicarboxylic acid units.

13. A pigment composition which comprises:
a pigment component including carbon black in an amount of about 5-80% by weight, treated with a water-dispersible polyester or polyesteramide polymer having an aromatic dicarboxylic acid moiety, an aliphatic or cycloaliphatic glycol residue, an average, per molecule, of between five and eight sodiosulfo(SO₃⁻Na⁺) substituents on the aromatic dicarboxylic acid units, in an amount of about 20-95% by weight, a surfactant in an amount of about 0.1-25% by weight and ethoxylated glycol in an amount of from about 0.1-10% by weight, in the presence of 0.2-5.99% by weight of water.

14. The composition of claim 13, further comprising:
one of an ionic surfactant and a nonionic surfactant in an amount of from about 0.001-25% by weight.

15. A method of making a pigment composition comprising the steps of:
grinding a mixture containing a pigment including carbon black with one of a water-dispersible polyester or polyesteramide, or Polyvinylpyrolidone (PVP) or Cellulosic polymers that are natural or synthetic acrylic polymers, an alkalizing agent and a plasticizer in the presence of added water
grinding the mixture obtained on a mill, for a time sufficient to obtain a particle size of 0.5-0.15 mu, wherein:
at least one roller of the roller mill is cold; and
the other roller is heated to the glass transition temperature of the polymer.

16. A cosmetic formulation, comprising:
a first liquid phase, containing water, of 38.0-45.0% by weight;
a pigment phase, containing 5.0-8.0 by weight of mixed pigment chips and separate black iron oxide;
said pigment chips, comprising:
a pigment component from 85.0-95.0% by weigh, further comprising:
cabon black being 35.0-45.0% by weight and polyester being 45.0-55.0% by weight;
a dispersant component from 1.5-3.0% by weight; and
a sodium hydroxide 50% solution from 0.25-0.75% by weight of the pigment phase;
a copolymer phase, of 25.0-30.0% by weight;
said copolymer phase, comprising at least the following:
propylene glycol monosterate about 2.0% by weight;
steric acid about 3.0% by weight; and
bees wax about 2.0-4.0% by weight;
a second liquid phase, containing 0.01-0.50% by weight preservative; and
a film forming phase, containing 15.00-21.00% by weight a water-based acrylic polymer dispersion with a hydrophobic character.

17. A cosmetic formulation, according to claim 16, wherein:
said first liquid phase, further comprising:
water 40.0-41.0% by weight;
HEC HHR 0.10-0.50% by weight;
PVP 0.50-1.50% by weight;
Methyl paraben 0.10-0.40% by weight; and
TEA 99% at 1.50-2.50% by weight.

18. A cosmetic formulation, according to claim 17, wherein:
said preservative in said second liquid phase is Imidazolidinyl urea.
